(19) United States
(12) Patent Application Publication
(10) Pub. No.: US 2006/0177484 A1
(43) Pub. Date: Aug. 10, 2006

(54) Cheese alternative product and process for preparing a cheese alternative product

(76) Inventors: Stephen May, Surrey (GB); Nigel Kenneth Harray, Sutton Coldfield (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/539,934

(22) PCT Filed: Oct. 30, 2003
(86) PCT No.: PCT/GB03/04704

§ 371 Date: Feb. 17, 2006

(30) Foreign Application Priority Data
Dec. 20, 2002 (GB) 02298750.5

(51) Int. Cl. A61K 47/00; A61K 36/18

(52) U.S. Cl. 424/439; 424/776

(57) ABSTRACT

A cheese alternative product is prepared from filled milk formed from dairy origin skimmed milk and vegetable oil. The vegetable contains blood cholesterol lowering agent in an amount such that in the product the blood cholesterol lowering agent has a blood cholesterol level lowering effect. The blood cholesterol lowering agent is phytosterol, oryzanol, tocopherol, tocotrienol, polyphenol or a mixture of any two or more thereof.

The present invention relates to a cheese alternative product and a process for preparing a cheese alternative product.

It is well established that ingestion of saturated fat can increase blood plasma total cholesterol concentration, and that this concentration is correlated with the extent of atherosclerosis and the incidence of coronary heart disease.

There has, therefore, been significant interest among consumers in food products which lower blood cholesterol levels. To this end, WO 96/38047 describes “healthy” spreads and a dressing formed from oryzanol-enriched refined sunflower oil, oryzanol being found to lower blood cholesterol levels. WO 96/38047 also suggests that phytosterols, tocopherols and tocotrienols can be used to lower cholesterol levels.

The present invention is at least partly based on the realisation that the benefits of blood cholesterol lowering compounds can also be obtained in foodstuffs, such as cheese, that usually are so high in dairy origin fats. (which in turn are high in saturated fats) as to be considered “unhealthy” for consumers at risk from high cholesterol levels.

As used herein, by “blood cholesterol lowering agent” we mean phytosterol, oryzanol, tocopherol, tocotrienol, polyphenol or a mixture of any two or more thereof.

In general terms the present invention provides a cheese alternative product containing a blood cholesterol lowering agent in an amount sufficient to have a blood cholesterol level lowering effect. That is, the blood cholesterol lowering agent should be present in an amount sufficient to partly or wholly counteract the blood cholesterol increasing effect of any saturated fat in the product. However, preferably the blood cholesterol lowering agent also has a general effect such that ingestion of the product decreases the blood plasma total cholesterol concentration.

Oryzanol consist of a mixture of ferulic acid esters of unsaturated triterpene alcohols and is also referred to as gamma-oryzanol. Only the term oryzanol is used herein. Within the group of tocopherols and tocotrienols one can distinguish alpha, beta, gamma and delta tocopherols and tocotrienols depending on the number and position of the methyl substituents on the chromane ring of the molecule. The terms tocopherol and tocotrienol are used herein to cover this whole family of molecules. For a further description and schematic drawings of oryzanol, tocopherol and tocotrienol according to the present invention, reference is made to “Separation of Vitamin E and gamma-Oryzanols from Rice Bran by Normal-phase Chromatography”, M. Diack and M. Saska, JAOCS. Vol. 71, no. 11, pp. 1211.

Phytosterols, also known as plant sterols or vegetable sterols can be classified in three groups, 4-desmethylsterols, 4-monomethylsterols and 4,4′-dimethylsterols. In oils they mainly exist as free sterols and sterol esters of fatty acids, although sterol glucosides and acylated sterol glucosides are also present. There are three major phytosterols namely beta-sitosterol, stigmasterol and campesterol. Schematic drawings of phytosterols according to the present invention are given in “Influence of Processing on Sterols of Edible-Vegetable Oils”, S. P. Kochhar, Prog. Lipid Res. 22: pp. 161-188. The term phytosterol as used herein covers the whole group of free phytosterols, phytosterol fatty acid esters and (acylated) phytosterol glucosides.

Polyphenols (or phenolics) can be defined chemically as substances which possess an aromatic ring bearing one or more hydroxy substituents, including functional derivatives. Reference is made-to “Phenolic Compounds in Food”, Chi-Tang Ho; Phenolic Compounds in Food and Their Effects on Health II, Am. Chemical Soc., 1992. As used herein, the term polyphenol refers to all plant phenolic molecules derived from a plant source with an antioxidant activity and not covered by the terms oryzanol, tocotrienol and tocopherol, e.g. simple phenols and phenolic acids, hydroxycinnamic acid derivatives (e.g. coumaric and ferulic acid) and flavonoids.

In a first aspect, the present invention provides a cheese alternative product prepared from filled milk formed from dairy origin skimmed milk and vegetable oil, wherein the vegetable oil contains blood cholesterol lowering agent in an amount such that in the product the blood cholesterol lowering agent has a blood cholesterol level lowering effect. As used herein, the term “filled milk” pertains to a water-continuous composition containing oil of a non-dairy origin, i.e. not derived from a milk product.

Advantageously, the vegetable oil not only introduces the blood cholesterol lowering agent into the product, but also displaces a significant amount of “unhealthy” dairy origin fat from the product (e.g. allows skimmed milk to be used in place of bovine fat milk).

Preferably the blood cholesterol lowering agent is phytosterol.

The vegetable oil may be rice bran oil, wheat germ oil, maize germ oil, oat oil, oat bran oil, sesame seed oil, soybean oil, sheanut oil, palm oil, rape oil or a mixture of any two or more thereof. Preferably, the oil may be rice bran oil, wheat germ oil, maize germ oil, oat oil, oat bran oil, sesame seed oil, soybean oil, sheanut oil or a mixture of any two or more thereof, as these oils all contain significant amounts of naturally-occurring phytosterol. More preferably the vegetable oil is wheat germ oil and/or rice bran oil.

In one embodiment, the filled milk contains only the skimmed milk and the vegetable oil. The milk from which the cheese is formed is then an entirely “natural” product, which should increase the consumer acceptability of the product. However, this does not exclude that in other embodiments the vegetable oil may be fortified by “non-natural” blood cholesterol lowering agent, i.e. agent that is derived, for example, via an isolating, refining and/or chemical process.

Preferably the filled milk does not contain an emulsifier.

The cheese alternative product preferably has a total fat content of at least 30 wt. %, and may have a total fat content of at least 40 wt. % or at least 45 wt. %. The cheese alternative product therefore retains the taste and mechanical properties of cheese having similar fat contents.

Preferably the total bovine fat content of the cheese alternative product is at most 20 wt. %. The total bovine fat content of the cheese alternative product is more preferably at most 10 wt. %, 5 wt. % or 1 wt. %. Reducing the bovine fat content may reduce the amount of blood cholesterol lowering agent required to produce a blood cholesterol lowering effect.

The cheese alternative product may contain at least 0.1 wt. % of blood cholesterol lowering agent, the weight percentage being based on the total weight of the cheese alternative product. We have found that relatively low amounts of blood cholesterol lowering agent in cheese products can be beneficial because the average daily intake of such products tends to be high. In contrast, spreads and similar products are generally consumed in smaller daily amounts, and so the amount of blood cholesterol lowering agent that is sometimes added to these products usually has to be higher.

However, in other embodiments the cheese alternative product contains at least 0.2, 0.5, 1 or 2 wt. % of blood cholesterol lowering agent.

Preferably the cheese alternative product contains at most 10 wt. % of blood cholesterol lowering agent, and more preferably at most 5 or 3 wt. %.

The product may be a cheese alternative to any of a wide variety of cheeses e.g. gouda, mozzarella, Roquefort, cheddar, stilton, emmental etc. However, preferably the product is a hard cheese alternative, such as a gouda or cheddar alternative.

A further aspect of the invention provides a process for preparing a cheese alternative product containing blood cholesterol lowering agent (which is preferably phytosterol) in an amount sufficient to have a blood cholesterol level lowering effect, the process comprising:

mixing vegetable oil with dairy origin skimmed milk to form filled milk, the vegetable oil containing blood cholesterol lowering agent,

forming curd and whey by adding a starter culture and/or a coagulant to the filled milk, and

separating the whey from the curd.

The curd can then be processed in a number of easy to obtain different styles of cheese alternative product, e.g. cheddar-style, gouda-style, emmental-style, stilton-style, mozzarella-style etc.

Preferably, the mixing is performed by injecting the vegetable oil into the skimmed milk to atomise and disperse the vegetable oil. This can allow a fine dispersion of vegetable oil to be formed without the use of an emulsifier.

Filled milks can be formed by-mechanical agitation of skimmed milk and non-dairy origin oil, a purpose of the agitation being to reduce the oil droplet size and to form a stable oil in water emulsion. However, agitation can destroy or damage the milk proteins, such as casein, essential for cheese production. We have found that injection atomising the vegetable oil allows a fine and homogeneous dispersion of vegetable oil droplets to be produced in the skimmed milk without significant damage to the skimmed milk casein. Furthermore, the oil thus dispersed appears to have an improved stability, such that problems of oil “sweating” from cheese alternatives obtained by the process are reduced or avoided. Also the thermal melting behaviour of the cheese alternative can be substantially similar to that of the corresponding “conventional” cheese produced without addition of vegetable oil.

Another aspect of the invention provides a cheese alternative product obtained by the process of the previous aspect. For example, the process may be used to obtain a cheese alternative product of the first aspect of invention.

The invention is illustrated by means of the following example and study which relate to cheddar alternatives.

### EXAMPLE

Raw milk used to produce the cheese alternative product was stored at less than 6° C. The raw milk was preheated to 55° C. and introduced into a centrifugal separator which separated the raw milk into 40 vol. % fat cream and 0.05 vol. % skimmed milk.

Refined wheat germ oil, which is naturally high in phytosterols, was heated to 45° C. and then injected into the skimmed milk. The injection system comprised a 50 mm diameter tube along which the skimmed milk (now also at 45° C.) was passed at a pressure of 1.5 bar and a flow rate of approximately 21,000 litres per hour. Four to six injection nozzles of 0.7 mm diameter formed in the wall of the tube allowed the wheat germ oil to be radially injected into the skimmed milk stream at a pressure in the range 200-220 bar. The nozzle orifices were covered with brass gauze to prevent any debris blocking the nozzle exits.

With this system the oil was injected at between 700 to 1,000 litres per hour, which produced a filled milk having 4 vol. % total fat. The injection caused the oil to atomise and form a stable emulsion with a consistent and small oil globule size.

Next, the filled milk was held at 73° C. for more than 20 seconds to pasteurise milk. The milk was cooled to 31° C. and divided into 15000 kg batches to fill respective cheesemaking vats. 3×500 g of mesophilic dairy starter culture was added to each batch, which was then allowed to ripen for 45 minutes to acidify the cheesemilk and generate flavour in the finished product.

1.25 kg of microbial rennet was added to each batch and the cheesemilk agitated for 3 minutes. The milk was left to coagulate and to develop flavour for 55 minutes. The coagulum was then cut with vertical knives to produce solid curd and liquid whey. Whilst being stirred, steam was applied to the vat jacket to increase the temperature to between 37 and 39.5° C. Stirring continued for a further ten minutes at this temperature and the vat contents then emptied onto perforated cooling/cheddaring tables.

Liquid whey drained off through the perforations and the curd was further agitated and squeezed until a titratable acidity of 0.4 vol. % lactic acid in the whey was reached, whereupon the curd was milled to thumbnail size. Salt was added to the curd and the salted curd used to fill cloth. lined stainless steel moulds. These were pressed at 60-100 psi overnight to expel further moisture and obtain a regular block shape. Finally, each block was held at 8 to 12° C. for 6 to 12 weeks to mature.

At the start of the cheesemaking process each vat contained:
14400 kg skimmed milk,
600 kg wheatgerm oil,
1.5 kg dairy culture, and
1.25 kg coagulant.

Proportionately, about 43 kg of salt was subsequently added.

From this about 1580 kg of finished cheese alternative product was obtained having good oil retention, meltability, texture and flavour, containing:
total fat content=32 to 36 wt. %,
moisture level=34.5 to 38.5 wt. %,
salt=1.65 to 2.15 wt. %, and
phytosterol=0.2 wt. %
   and having a pH in the range 5.15 to 5.45 pH.

The finished cheese alternative product was also found to have good mechanical properties; grating, slicing and repressing well.

Study

A study was performed into the effect on blood cholesterol levels of a cheese alternative according to the present invention containing wheatgerm oil.

Materials and Methods

28 male volunteers (age 50-66 years, BMI 23-32 Kg/m²) participated in a three-week intervention study to investigate the effect of sterols and polyunsaturated fats on plasma cholesterol levels. Subjects were assigned, in a paired design, to one of either two groups; control (cheese alternative comparative example formed from vegetable oil blend containing low levels of sterols and polyunsaturated fats) or intervention group (cheese alternative according to the present invention containing significant sterol levels/high polyunsaturated fats). Both groups ate approximately 65 g of the respective cheese alternative per day. Venous blood was collected at baseline and each week thereafter to assess cholesterol levels. Subjects completed a sensory evaluation on both cheese alternatives.

Results

On average, from week 0 to week 3, total cholesterol significantly decreased by 5.7% in the intervention group (P=0.022; 98% confidence level) with no change seen in the control group. Both groups saw LDL cholesterol drop by approximately 12%. Tryglycerides remained unaltered in both groups.

There was no difference in the subjects' preference, subjectively rated, relating to pleasantness and palatability of the two cheese alternatives.

### Conclusion

The daily consumption of 65 gms of cheese alternative containing sterols and high levels of polyunsaturated fats (each 65 gms sample in the trials contained approximately 0.2 gms of sterols and 10 gms of polyunsaturated fats) significantly reduced plasma cholesterol in mildly hypercholesterolaemic male subjects, within a 3-week period. Lipid profile was favourably altered, reducing LDL (bad) cholesterol.

The statistical analysis of the results indicates that (a) the results are no chance occurrence—the reduction in blood cholesterol is clearly due to the cheese, and (b) of every 100 people eating the cheese alternative under study, on average, 98 would have their cholesterol lowered by at least 5.7%.

1-7. (canceled)

8. A cheese alternative product prepared from filled milk formed from dairy origin skimmed milk and vegetable oil, wherein
the vegetable oil contains blood cholesterol lowering agent in an amount such that in the product the blood cholesterol lowering agent has a blood cholesterol level lowering effect, and
the blood cholesterol lowering agent is phytosterol, oryzanol, tocopherol, tocotrienol, polyphenol or a mixture-of any two or more thereof.

9. A cheese alternative product according to claim 8 containing at least 0.1 wt. % of blood cholesterol lowering agent.

10. A cheese alternative product according to claim 8 wherein the blood cholesterol lowering agent is phytosterol.

11. A cheese alternative product according to claim 9 wherein the blood cholesterol lowering agent is phytosterol.

12. A process for preparing a cheese alternative product containing blood cholesterol lowering agent in an amount sufficient to have a blood cholesterol level lowering effect, the process comprising:
mixing vegetable oil with dairy origin skimmed milk to form filled milk, the vegetable oil containing blood cholesterol lowering agent,
forming curd and whey by adding a starter culture and/or a coagulant to the filled milk, and
separating the whey from the curd;
wherein the blood cholesterol lowering agent is phytosterol, oryzanol, tocopherol, tocotrienol, polyphenol or a mixture of any two or more thereof.

13. A process according to claim 12, wherein the mixing is performed by injecting the vegetable oil into the skimmed milk to atomise and disperse the vegetable oil.

14. A process according to claim 12, wherein the blood cholesterol lowering agent is phytosterol.

15. A process according to claim 13, wherein the blood cholesterol lowering agent is phytosterol.

16. A cheese alternative product obtained by the process of claim 12.

17. A cheese alternative product obtained by the process of claim 13.

18. A cheese alternative product obtained by the process of claim 14.

19. A cheese alternative product obtained by the process of claim 15.
